(19) United States
(12) Patent Application Publication
(10) Pub. No.: US 2023/0067679 A1
(43) Pub. Date: Mar. 2, 2023

(54) FUNCTIONALISATION OF 1,3-ALPHA-DIENES (II)

(71) Applicant: DSM IP Assets B.V., (US)

(72) Inventors: Werner BONRATH, Kaiseraugst (CH); Felix IMBERI, Kaiseraugst (CH); Marc-André MUELLER, Kaiseraugst (CH); Bettina WUESTENBERG, Kaiseraugst (CH)

(21) Appl. No.: 17/787,794

(22) PCT Filed: Dec. 15, 2020
(86) PCT No.: PCT/EP2020/086177

§ 371 Date: Jun. 21, 2022

(30) Foreign Application Priority Data
Dec. 23, 2019 (EP) 19219363.9

(51) Int. Cl. C07C 403/08

(52) U.S. Cl. CPC C07C 403/08; C07C 2601/16

(57) ABSTRACT

The present invention relates to the functionalisation of specific 1,3-alpha-dienes (by hydroboration). These functionalized 1,3-alpha-dienes are important intermediates in organic synthesis (especially in the synthesis of carotenoids, vitamin A and/or vitamin A derivatives).

The present invention relates to the functionalisation of specific 1,3-alpha-dienes. These functionalized 1,3-alpha-dienes are important intermediates in organic synthesis (especially in the synthesis of carotenoids, vitamin A and/or vitamin A derivatives).

The goal was to find intermediates in the synthesis of carotenoids, vitamin A and/or vitamin A derivatives and an easy and effective way to produce them.

The present invention relates to the functionalisation of specific 1,3-alpha-dienes by hydroboration followed by an oxidation to the corresponding alcohols.

Hydroboration is a well-known reaction from the prior art.

The obtained product, which is a terminal alcohol is a very interesting intermediate in the organic synthesis, especially in the synthesis of carotenoids, vitamin A and vitamin A derivatives.

Therefore, there is a need for a hydroboration process, which allows to produce specific and important terminal alcohols in an excellent yield.

Therefore, the present invention relates to a process (P), wherein a compound of formula (I)

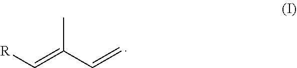

wherein R is

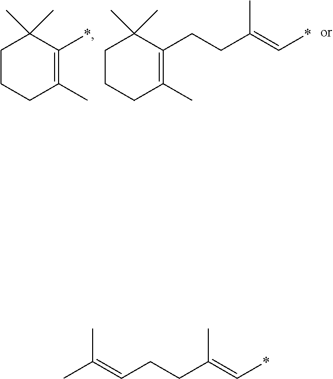

(wherein the asterix shows the connecting bond) is reacted with a borane tetrahydrofuran complex and then oxidized to the corresponding alcohol.

The obtained alcohol has the following formula (II)

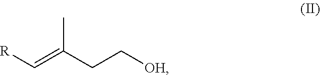

wherein R has the same means as in compound of formula (I).

The hydroboration can be carried out with or without any solvent. In case a solvent is used, the solvent needs to be inert. Usually THF is used or a mixture of THF with at least one other inert solvent (such as i.e. cyclohexene).

Preferably, the hydroboration is carried in an inert solvent.

Therefore, the present invention also relates to a hydroboration process (P1), which is the hydroboration process (P), wherein the process is carried out in an inert solvent.

Therefore, the present invention also relates to a h hydroboration process (P1′), which is the hydroboration process (P1), wherein the process is carried out in THF and optionally at least one other solvent.

Preferred is a process wherein the compound of formula (Ia)

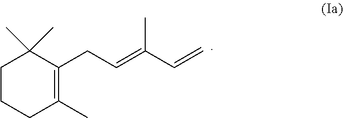

is used as starting material.

Also preferred is a process wherein the compound of formula (Ib)

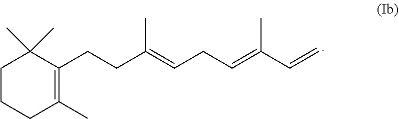 [SMILES: C=C/C(C)=C/C/C=C(\C)CCC1=C(C)CCCC1(C)C]

is used as starting material.

Also preferred is a process wherein the compound of formula (Ic)

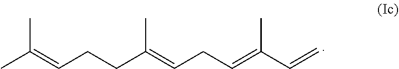

is used as starting material.

Therefore, the present invention also relates to a hydroboration process (P2), which is the hydroboration process (P), (P1) or (P1′), wherein the compound of formula (Ia)

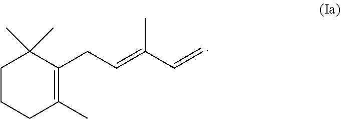

is used as starting material.

Therefore, the present invention also relates to a hydroboration process (P3), which is the hydroboration process (P), (P1) or (P1′), wherein the compound of formula (Ib)

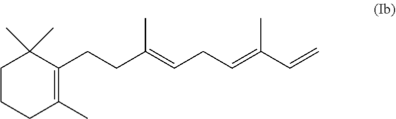 [SMILES: C=C/C(C)=C/C/C=C(\C)CCC1=C(C)CCCC1(C)C]

is used as starting material.

Therefore, the present invention also relates to a hydroboration process (P4), which is the hydroboration process (P), (P1) or (P1′), wherein the compound of formula (Ic)

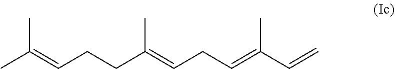

is used as starting material.

Furthermore, the compound of formula (IIb), which is obtained alcohol when the compound formula (Ib) is used as starting material,

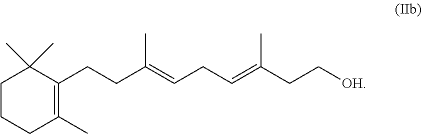 [SMILES: CC1=C(CC/C(C)=C/C/C=C(\C)CCO)C(C)(C)CCC1]

is new.

Therefore, the present invention also relates to the compound of formula (IIb)

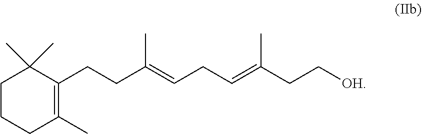 [SMILES: CC1=C(CC/C(C)=C/C/C=C(\C)CCO)C(C)(C)CCC1]

The starting material used in the process according to the present invention can be obtained by commonly known processes.

Cyclo-alpha-farnesene (compound of formula (Ia) can be prepared by literature-known procedures (Desai, Shailesh R. et al, Tetrahedron 1992, 48(3), 481-90) in 52.5% over-all yield.

This is done according to the following way:

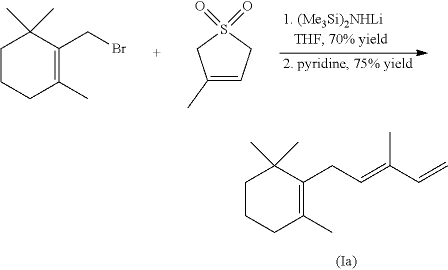

Alternatively, the compound of formula (Ia) can be produced by using the commercially available (2E)-2-methyl-4-(2,6,6-trimethyl-1-cyclohexen-1-yl)-2-butenal via Wittig reaction:

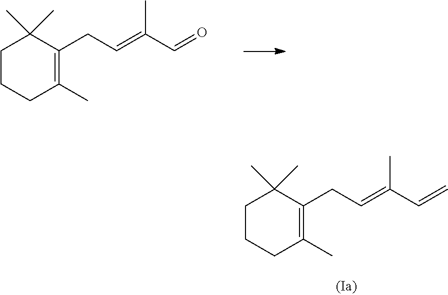

The compound of formula (Ib), which is not known from any prior art, can be produced as follows:

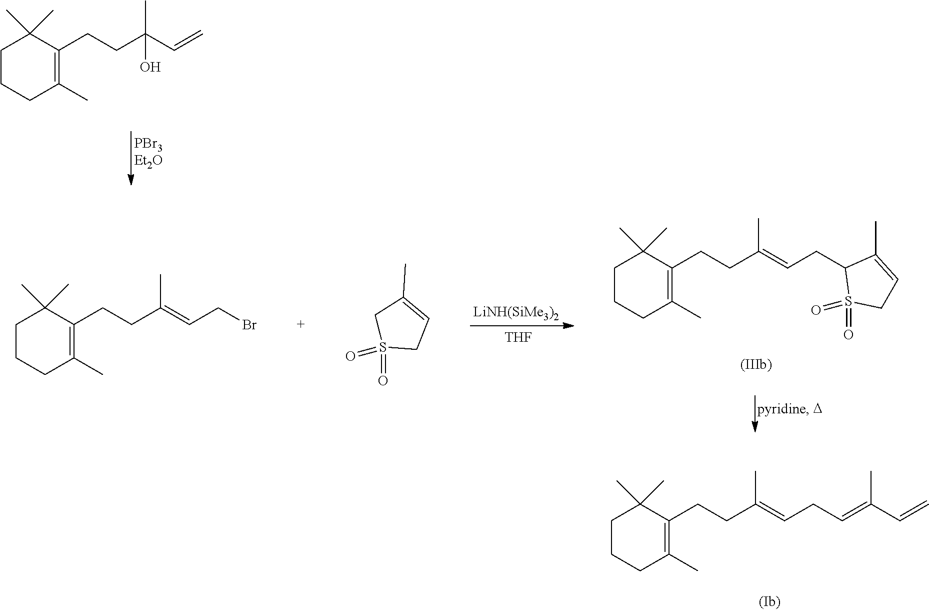

The compound of formula (IIIb) is also a new compound.

The compound of formula (Ic) is produced as follows (according to Synth. Commun. 1990, 20(4), 523-533; J. Agric. Food Chem. 2016, 64, 6809-6818):

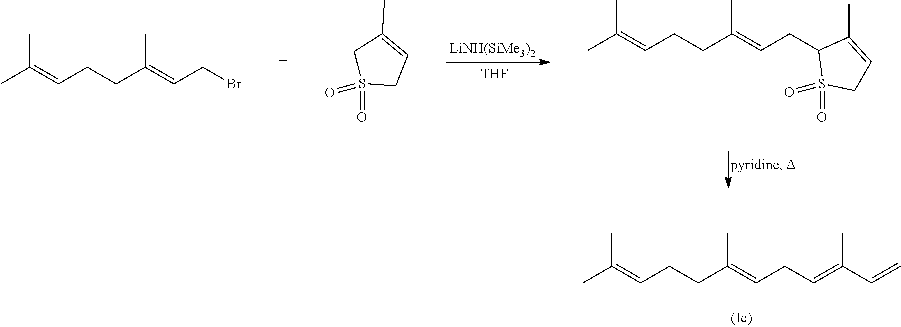

The borane tetrahydrofuran complex (BH3-THF complex or borane-tetrahydrofuran), which is commercially available, is added to the reaction mixture in equimolar (or slight excess) in regard to the compound of formula (I). Preferably, the borane tetrahydrofuran complex is added in a slight excess (between 1.1-2 eq) in regard to the compound of formula (I).

Therefore, the present invention also relates to a hydroboration process (P5), which is the hydroboration process (P), (P1), (P1′), (P2), (P3) or (P4), wherein the borane tetrahydrofuran complex is added to the reaction mixture in an equimolar amount in regard to the compound of formula (I).

Therefore, the present invention also relates to a hydroboration process (P5′), which is the hydroboration process (P), (P1), (P1′), (P2), (P3) or (P4), wherein the borane tetrahydrofuran complex is added in a slight excess (between 1.1-2 eq) in regard to the compound of formula (I).

The hydroboration reaction is usually carried out at temperature range of from −10° C.-30° C. Preferred are elevated temperatures (from −5° C. to 25° C.).

Therefore, the present invention also relates to a hydroboration process (P6), which is the hydroboration process (P), (P1), (P1′), (P2), (P3), (P4), (P5) or (P5′), wherein the process is carried out at temperature range of from −10° C.-30° C.

Therefore, the present invention also relates to a hydroboration process (P6′), which is the hydroboration process (P6), wherein the process is carried out at temperature range of from −5° C. to 25° C.

Furthermore, the hydroboration reaction can be carried out under an inert gas atmosphere (usually N₂ gas).

Therefore, the present invention also relates to a hydroboration process (P7), which is the hydroboration process (P), (P1), (P1′), (P2), (P3), (P4), (P5), (P5′), (P6) or (P6′), wherein the process is carried out under an inert gas atmosphere (usually N₂ gas).

To obtain the intermediates, which are very suitable in the organic synthesis (especially in the production of carotenoids, vitamin A and vitamin A derivatives, the reaction products of the hydroboration process (the compounds of formula (III) and (III′)) are converted into alcohols via an oxidative cleavage

The oxidative cleavage is carried according to well-known processes. Usually and preferably the oxidative cleavage is carried in the presence of hydrogen peroxide and a base.

Therefore the present invention also relates to a hydroboration process (P8), which is the hydroboration process (P), (P1), (P1′), (P2), (P3), (P4), (P5), (P5′), (P6) or (P6′) or (P7), wherein in a second step the reaction product is converted into the alcohols via an oxidative cleavage in the presence of hydrogen peroxide and a base.

The following example illustrate the invention. All parts are related to weight and the temperatures are given in ° C.

### EXAMPLES

### Example 1

In a 25-ml flask under inert gas atmosphere, borane tetrahydrofuran complex (1M, 1.666 ml, 1.666 mmol) was cooled to 0° C. Cyclohexene (0.169 ml, 1.666 mmol) in THF dry (4.00 ml) was added within 5 min. After 10 min a turbid white reaction mixture was obtained and stirring was continued for 1 h at 0° C. Then, cyclo-α-farnesene (compound of formula (Ia)) (200 mg, 0.833 mmol) in THF dry (2.00 ml) was added. The reaction mixture was allowed to warm to room temperature and was monitored by HPLC. After 1 h 15 min full conversion was observed. Subsequently sodium hydroxide (5.00 ml, 9.99 mmol) and hydrogen peroxide (30%, 0.595 ml, 5.83 mmol) were added and stirring was continued for another hour. Then, the reaction mixture was diluted with diethyl ether (20 mi), transferred to a separation funnel and washed with deionized water. The layers were separated, and the organic layer was washed with brine. The aqueous layers were re-extracted with diethyl ether. The combined organic layers were dried over magnesium sulfate, filtered and evaporated under reduced pressure. The crude product (compound of formula (IIa)) was obtained as colorless oil (263 mg, content by qNMR: 56.4 wt %, 100% conversion, 80.0% yield).

### Example 2

In a 25-ml flask under inert gas atmosphere, borane tetrahydrofuran complex (1M, 1.869 ml, 1.869 mmol) was cooled to 0° C. Cyclohexene (0.190 ml, 1.869 mmol) in THF dry (4.50 ml) was added within 5 min. After 10 min a turbid white reaction mixture was obtained and stirring was continued for 1 h at 0° C. Then, α-farnesene (compound of formula (Ic)) (200 mg, 0.935 mmol) in THF dry (2.25 ml) was added. The reaction mixture was allowed to warm to room temperature. After 2.5 hours, subsequently sodium hydroxide (5.61 ml, 11.22 mmol) and hydrogen peroxide (30%, 0.668 ml, 6.54 mmol) were added and stirring was continued for another hour. Then, the reaction mixture was diluted with diethyl ether (20 ml), transferred to a separation funnel and washed with deionized water. The layers were separated, and the organic layer was washed with brine. The aqueous layers were re-extracted with diethyl ether. The combined organic layers were dried over magnesium sulfate, filtered and evaporated under reduced pressure. The crude product (compound of formula (IIc)) was obtained as colorless oil (416 mg, content by qNMR: 42.0 wt %, 91.9% conversion, 84.0% yield).

### Example 3

In a 25-ml flask under inert gas atmosphere, borane tetrahydrofuran complex (1M, 2.433 ml, 2.433 mmol) was cooled to 0° C. Cyclohexene (0.345 ml, 3.41 mmol) in THF dry (6.25 ml) was added within 5 min. After 15 min a turbid white reaction mixture was obtained and stirring was continued for 1 h at 0° C. Then, 1,3-diene (compound or formula (Ib)) (500 mg, 1.703 mmol) in THF dry (2.50 ml) was added. The reaction mixture was allowed to warm to room temperature. After 1.5 hours, subsequently sodium hydroxide (5.11 ml, 10.22 mmol) and hydrogen peroxide (30%, 0.609 ml, 5.96 mmol) were added and stirring was continued for another hour. Then, the reaction mixture was diluted with diethyl ether (20 ml), transferred to a separation funnel and washed with deionized water. The layers were separated, and the organic layer was washed with brine. The aqueous layers were re-extracted with diethyl ether. The combined organic layers were dried over magnesium sulfate, filtered and evaporated under reduced pressure. The crude product (compound of formula (IIb) was obtained as colorless oil (892 mg, content by qNMR: 36.4 wt %, 90.3% conversion, 66.4% yield).

1. A process, wherein a compound of formula (I) 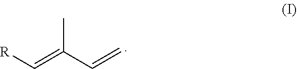
wherein R is 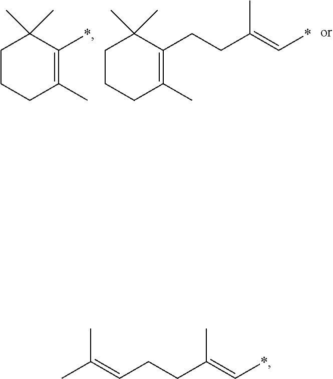
(wherein the asterix shows the connecting bond) is reacted with a borane tetrahydrofuran complex and then oxidized to the corresponding alcohol.

2. Process according to claim 1, wherein the process is carried out in an inert solvent.

3. Process according to claim 1, wherein the process is carried out in THF and optionally at least one other solvent.

4. Process according to claim 1, wherein the compound of formula (Ia) 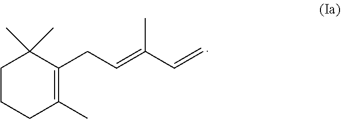
is used as starting material.

5. Process according to claim 1, wherein the compound of formula (Ib) 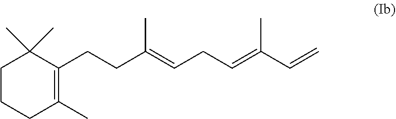 [SMILES: C=C/C(C)=C/C/C=C(\C)CCC1=C(C)CCCC1(C)C]
is used as starting material.

6. Process according to claim 1, wherein the compound of formula (Ic) 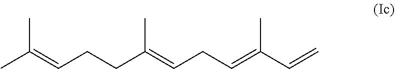
is used as starting material.

7. Process according to claim 1, wherein the borane tetrahydrofuran complex is added to the reaction mixture in an equimolar amount in regard to the compound of formula (I).

8. Process according to claim 1, wherein the borane tetrahydrofuran complex is added in a slight excess (between 1.1-2 eq) in regard to the compound of formula (I).

9. Process according to claim 1, wherein the process is carried out at temperature range of from −10° C.-30° C.

10. Process according to claim 1, wherein the process is carried out at temperature range of from −5° C. to 25° C.

11. Process according to claim 1, wherein the process is carried out under an inert gas atmosphere (usually N₂ gas).

12. Process according to claim 1, wherein in a second step the reaction product is converted into the alcohols via an oxidative cleavage in the presence of hydrogen peroxide and a base.

13. Compound of formula (IIb) 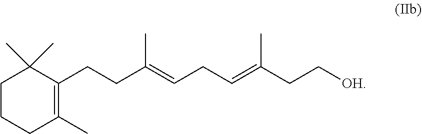 [SMILES: CC1=C(CC/C(C)=C/C/C=C(\C)CCO)C(C)(C)CCC1]
